(19) United States
(12) Patent Application Publication
(10) Pub. No.: US 2008/0081069 A1
(43) Pub. Date: Apr. 3, 2008

(54) NOVEL CONTROLLED RELEASE FORMULATIONS OF DIVALPROEX SODIUM

(75) Inventors: Ramprasad Prasad, Pune (IN); T. Vijaya Kumar, Pune (IN); Himadri Sen, Pune (IN)

Correspondence Address: MERCHANT & GOULD PC, P.O. BOX 2903, MINNEAPOLIS, MN, 55402-0903, US

(73) Assignee: Lupin Limited, Mumbai (IN)

(21) Appl. No.: 11/863,667

(22) Filed: Sep. 28, 2007

(30) Foreign Application Priority Data
Sep. 28, 2006 (IN) 1010KOL/2006

(51) Int. Cl. A61K 9/22; A61K 31/19; A61K 31/35; A61P 43/00; A61K 31/53; A61K 9/14

(52) U.S. Cl. 424/468; 424/489; 514/242; 514/459; 514/557

(57) ABSTRACT

A controlled release formulation comprising less than about 40% of anti-epileptic drug, about 20% to about 50% of rate controlling polymer and silica having a particle size less than about 1 micron and specific surface area not less than 70 m²/g, all weight percentages are based upon the total weight of the dosage form, manufactured under normal atmospheric conditions.

### FIELD OF THE INVENTION

The present invention addresses novel controlled release formulation of anti-epileptics. The controlled release dosage formulation releases the drug in small amounts throughout the pre-determined time period and thus maintains the drug level in the blood within a narrow therapeutically effective range.

### BACKGROUND OF THE INVENTION

Anti-epileptic drug are vital in preventing fits for people with epilepsy, thereby greatly enhancing quality of life. Drugs such as Valproic acid, sodium valproate, valpromide, divalproex sodium, lamotrigine, topiramate, carbamazepine, gabapentin, felbamate and the like are the first line therapy for the treatment of epilepsy.

Anti-epileptic drugs (AEDs) are also promising agents for the prevention of migraine and other head pain. Migraine and epilepsy share several clinical features and respond to many of the same pharmacological agents, suggesting that similar mechanisms may be involved in their pathophysiology.

The most common side effects associated with epilepsy medicines are drowsiness, irritability, nausea, rash, and clumsiness. Some drugs produce changes in emotions, memory or behavior, or affect learning. Controlled release formulations for anti-epileptic drugs were prepared, which provides reduction in the above mentioned side effect and along with it increases patient compliance, avoids a typical peak-valley plasma concentration profile and fluctuations in the drug concentration and ultimately reduction in the precipitation of adverse effects especially of a drug with a narrow therapeutic index whenever overmedication occurs.

Some of the anti-epileptic drugs such as valproic acid, sodium valproate, valpromide, and divalproex sodium dissociate to valproate ion within the gastrointestinal tract, which on absorption produces the desired therapeutic effect.

Divalproex sodium is a singe crystalline entity consisting of one molecule each of valproic acid and sodium valproate. Valproic acid is a liquid and sodium valproate although solid is very hygroscopic. Valproic acid and its derivatives are either liquid or liquefy rapidly and become sticky. Further most of them are hygroscopic in nature. These physicochemical properties pose serious problems during manufacture of pharmaceutical composition.

It is an effective anti-epileptic anti-migraine drug and is also used in bipolar disorders. The active moiety of divalproex sodium is valproate ion. Valproic acid and its derivatives have relatively short elimination half-life. Frequent dosing is essential which reduces patient compliance.

Divalproex sodium controlled release tablets are available as Depakote ER® in 250 mg and 500 mg strengths.

U.S. Pat. No. 4,913,906 assigned to Yissum describes controlled release dosage forms of valproic acid, its amide or any other pharmaceutically acceptable derivative of valproic acid, which upon administration to humans provides a serum level of valproic acid, in combination with an additive, which is selected from physiologically acceptable polymeric substances and from native proteins. The novel pharmaceutical compositions are prepared by applying a high pressure to a mixture of the ingredients. They result in a prolonged serum level of the active ingredient.

U.S. Pat. No. 5,019,398 assigned to Sanofi discloses a sustained-release tablet of divalproex sodium in a matrix of hydroxypropyl methylcellulose and hydrated silica.

U.S. Pat. No. 5,169,642 assigned to Abbott discloses a sustained release dosage form comprising granules of divalproex sodium or amides or esters of valproic acid coated with a sustained release composition comprising ethyl cellulose or a methacrylic methyl ester, a plasticizer, a detackifying agent, and a slow-release polymeric viscosity agent.

U.S. Pat. No. 6,287,598 assigned to Alza Corporation discloses a method for control of epilepsy by delivering a therapeutic composition of valproic acid or a derivative in combination with a poly(alkylene oxide).

U.S. Pat. No. 6,419,953 assigned to Abbott Laboratories discloses a hydrophilic matrix type controlled release tablet formulation containing about 50% to about 55% of valproate compound. The major problem according to the invention was of stickiness, which was overcome only by using a special grade silicon dioxide having a larger average particle size ranging from about 1 micron to about 10 microns.

U.S. Pat. No. 6,511,678 assigned to Abbott Laboratories disclose the controlled release formulation prepared by using about 40% to 80% of divalproex sodium. The formulation uses a particular size range of silicon dioxide to overcome the problem of stickiness and low bulk density.

US application 2004/0037880, applicant Ranbaxy, discloses controlled release formulations of a drug capable of dissociating to produce valproate ion. The problem of stickiness was avoided by manufacturing under controlled atmospheric conditions without using any special grade silicon dioxide. A controlled atmospheric condition according to the application has a temperature of form about 27° C. to about 35° C. and a relative humidity of less than 40%.

PCT application WO 2006/011001, applicant Wockhardt, discloses a controlled release tablet composition for once a daily administration of a valproate salt manufactured under controlled atmospheric condition (temperature less than about 27° C. and relative humidity above 40%) so that there is minimum sticking of the granule material to die and punch surfaces.

While a number of controlled releases divalproex sodium formulations are available but there remains a need for improved formulations of Divalproex sodium. The formulations of the prior art uses either a larger size silicon dioxide or controlled atmospheric conditions to manufacture controlled release formulations of divalproex which are free from processing problems. However, the formulations of the present invention are developed using silica having a smaller particle size manufactured under normal atmospheric conditions.

### OBJECTS OF THE INVENTION

One object of the present invention is to provide a novel and improved controlled release formulation of anti-epileptics comprising drug and a rate controlling polymer.

A further object of the present invention is to provide controlled release formulations of Divalproex sodium comprising about less than 40% of Divalproex Sodium and about 20% to about 50% of rate controlling polymer and Silica free of sticking problems encountered during manufacturing.

### SUMMARY OF THE INVENTION

According to one aspect of present invention there is provided controlled release formulation of Divalproex sodium comprising about less than 40% of Divalproex Sodium and about 20% to about 50% of rate controlling polymer and Silica free of sticking problems encountered during manufacturing

According to another aspect, the invention provides controlled release formulations of divalproex sodium free of processing problem, more particularly sticking or hardness or capping, comprising less than about 40% of divalproex sodium, about 20% to about 50% of rate controlling polymer, less than about 10% of silica having a smaller particle size, ranging from less than about 1 micron and having a specific surface area of not less than 70 m²/g.

According to a further aspect, the invention provides controlled release formulation of divalproex sodium having not less than 75% of drug released in 24 hours.

According to another aspect, the invention provides the process for the preparation of controlled release formulation of divalproex sodium.

### DETAILED DESCRIPTION OF INVENTION

The present invention provides a controlled release formulation of anti-epileptics.

Anti-epileptic drugs for the present formulation includes drugs which are used in the treatment and or prophylaxis of epilepsy and are but not limited to valproic acid, sodium valproate, valpromide, divalproex sodium, gabapentin, felbamate, lamotrigine, quetiapine, topiramate and the like.

The invention also relates to new and improved controlled release formulation of divalproex sodium.

Valproate compounds include all those compounds, which dissociates into valproate ion, which on absorption produce the desired therapeutic effect. Examples of valproate compounds are, valproic acid, sodium valproate, valpromide, divalproex sodium or the like. The most preferred compound of the present invention is divalproex sodium. Divalproex sodium is an effective anti-epileptic agent and is also used for migraine and bipolar disorders. It can be conveniently prepared as described in U.S. Pat. No. 4,988,731 and U.S. Pat. No. 5,212,326. Reference should be made to said patent for its full disclosure, the entire disclosure of which is incorporated herein by reference.

Controlled release formulation describes a formulation that does not release active drug substance immediately after oral dosing and that allows a reduction in dosage frequency. A controlled release formulation includes but is not limited to extended release, delayed release, sustained release formulations. A controlled release formulation may be administered once a day. Rate controlling polymer according to present invention includes agents, which controls the release of drug from the formulation.

An example of controlled release formulation is matrix formulation (erodable and non-erodable), diffusion controlled membrane coated or osmotic pumps.

The controlled release formulations of divalproex sodium, manufactured according to U.S. Pat. No. 6,419,953, uses silicon dioxide having a larger average particle size ranging from about 1 micron to about 10 micron to avoid the sticking and other processing formulations. Also WO 2004/0037880 uses controlled atmospheric conditions for the manufacture of divalproex sodium formulation to avoid the processing problems, more specifically sticking. In one aspect of the invention we have surprisingly found that controlled release formulations of divalproex sodium can be manufactured using smaller particle size from about less than about 1 micron and specific surface area not less than 70 m²/g which are free from processing problem, more particularly sticking, hardness or capping, encountered during pharmaceutical formulation development. Further the controlled release formulations of the present invention are manufactured under normal atmospheric conditions.

The normal atmospheric conditions under which the tablets are generally manufactured is at a temperature not more than about 25° C. and relative humidity of about 40% to about 55%. The present invention provides a controlled release formulation comprising from about 10% to about 80% of valproate compound, preferably less than about 40% of valproate compound and about 10% to about 60% of rate controlling polymer, all weight percentages are based upon the total weight of dosage form. Preferably the rate-controlling polymer is from about 20% to about 50%. The most preferable valproate compound is divalproex sodium.

In addition the pharmaceutical composition of the present invention may include silica. Silica used may be hydrophobic silica or hydrophilic silica or a combination of hydrophobic and hydrophilic silica. The formulation of the present invention may comprise of about 10% to about 80% of divalproex sodium, about 10% to about 60% of rate controlling polymer and less than about 10% of silica having a smaller particle size ranging from less than about 1 micron and specific surface area not less than 70 m²/g to avoid any processing problem, more particularly sticking or hardness or capping, encountered during pharmaceutical formulation development based on the total weight of the dosage form. Silica used may be added intra-granularly and/or extra-granularly along with other excipients. More preferably silica used is hydrophobic fumed silica commercially available as Aerosil. The most preferable hydrophobic fumed silica is Aerosil R972 and R972 Pharma grade from Degussa having an average primary particle size of 16 nm and specific surface area not less than 70 m²/g.

In addition to above the pharmaceutical composition of the present invention may include lubricant. Lubricants referred to in the present invention include one or more selected from those well known in the art, as exemplified can be Stearate, hydrogenated vegetable oil, sodium stearyl fumarate, talc, colloidal silicon dioxide, palmitic acid, carnauba wax, glyceryl monostearate, microcrystalline wax, polyoxyethylene monostearates, fats and stearic acid or mixtures thereof.

Oral rate controlling polymers that are used in the present formulation may include hydrophilic polymer, hydrophobic polymer or a combination of hydrophilic and hydrophilic polymer. Examples of suitable hydrophilic polymers include hydroxypropyl methylcellulose, hydroxypropyl cellulose, methylcellulose, vinyl acetate copolymers, polysaccharides as alginates, xanthan gum, Chitosan, carrageenan, dextran and the like, polyalkylene oxides as polyethylene oxide and the likes, methaacrylic acid copolymers, maleic anhydride/methyl vinyl ether copolymers and the like. Hydrophobic polymers include acrylates, cellulose derivatives as ethylcellulose, cellulose acetate and the likes, methaacrylates, high molecular weight polyvinyl alcohols, waxes and the likes. The polymers used can also be eroding or non-eroding or combination of both. The rate-controlling polymer used may range from about 10% to about 60%, most preferably in the range of about 20% to about 50%, based on the total weight of the dosage form.

The formulation will, in general comprise of one or more excipients. Examples of excipients include, but are not limited to, diluents, disintegrants, lubricant, glident, binders, fillers, surfactant, solubilizers, and wetting agents. A combination of excipients may also be used. Such excipients will include diluents such as mannitol, dextrose, xylitol, sorbitol, gelatin, acacia, sucrose, microcrystalline cellulose, calcium carbonate, calcium phosphate dibasic, calcium phosphate tribasic, calcium sulfate, lactose, starches, vinyl polymers and the likes, disintegrants referred to in the present invention include one or more of microcrystalline cellulose, croscarmellose sodium, crospovidone, carboxymethyl starch sodium, sodium starch glycolate and the likes, binders referred to in the present invention include one or more celluloses such as hydroxypropyl cellulose, hydroxy ethyl cellulose, ethyl cellulose, hydroxypropyl methyl cellulose, methyl cellulose or mixtures thereof, acrylates, methacrylates, povidone, sucrose, corn or maize starch, pregelatinized starch and the like, coloring agents such as ferric oxide, FD&C dyes, lakes and the likes and flavoring agent. Examples of glidents include but are not limited to silica, magnesium trisilicate, powdered cellulose, talc, and starch.

The pharmaceutical formulation of the present invention may also include less than about 5% of lactose.

The formulation of the present invention may be prepared by conventional techniques well known to those skilled in the art such as wet granulation, melt granulation, direct compression or dry compaction and/or slugging and the like. Most preferably the formulation of the present invention is prepared by dry compaction. The granules of the present invention comprise of about 10% to about 80% divalproex sodium, about 10% to about 60% of rate controlling polymer, based on the total weigh of the dosage form.

Most preferably the formulation and/or dosage form of the present invention may be a tablet or granules filled in capsule or pellet.

The dosage forms may be coated with one or more coatings, enteric or film coating as is well known in the art such as shellac, zein, hydroxypropylmethylcellulose, ethyl cellulose, polymethacrylates, polyvinyl acetate pthalate, cellulose acetate pthalate, triacetin, dibulty sebecate, a mixture of polyethylene glycol, titanium dioxide and the like.

The coating may be performed by conventional means using commercially available, ready-to-coat preparations, sold under various brand names such as various grades of Opadry®, Surelease® Dispersions or mixtures thereof and the like.

The dissolution of the present invention was carried out in type 2 dissolution apparatus, paddle, at 100 rpm, at a temperature of 37±0.5° C., in 500 ml of 0.1N HCl for 45 min, followed by 900 ml of 0.05M phosphate buffer containing 75 mM sodium lauryl sulfate, pH 5.5 for the remainder of the testing period and may release not more than about 40% of total valproate is released after 3 hours of measurement in said apparatus, from about 30% to about 70% of total valproate is released after 9 hours of measurement in said apparatus, from about 40% to about 90% of total valproate is released after 12 hours of measurement in said apparatus, not less than about 75% of total valproate is released after 24 hours of measurement in said apparatus.

The following examples are illustrative of the present invention, and the example should not be considered as limiting the scope of this invention in any way, as these examples and other equivalents thereof will become apparent to those versed in the art, in the light of the present disclosure, and the accompanying claims.

TABLE 1
Example 1

| Ingredients | Amount per tablet (%) |
|---|---|
| Divalproex sodium | 35 |
| HPMC K100 MP CR | 29 |
| Hydroxypropylcellulose | 14 |
| Lactose | 5 |
| Microcrystalline cellulose | 4 |
| Silica | 8 |
| Magnesium stearate | 2 |
| Coating | 3 |

TABLE 2
Example 2

| Ingredients | Amount per tablet (%) |
|---|---|
| Lamotrigene | 35 |
| HPMC K100 MP CR | 27 |
| Hydroxypropylcellulose | 15 |
| Lactose | 5 |
| Microcrystalline cellulose | 6 |
| Silica | 7 |
| Magnesium Stearate | 2 |
| Coating | 3 |

Procedure:
1. Active Drug was sifted
2. HPMC K00 MP CR, Lactose anhydrous and microcrystalline cellulose were sifted.
3. Step 1 and Step 2 was mixed in rapid mixer granulator.
4. Binder solution was prepared by dissolving HPC in purified water.
5. Step 3 was granulated with Step 4 and dried.

6. The Step 5 was lubricated with extra granular ingredients and compressed to get tablet and coated.

TABLE 3
Illustrative Examples

| Ingredients | Example 3 | Example 4 | Example 5 | Example 6 | Example 7 | Example 8 | Example 9 |
|---|---|---|---|---|---|---|---|
| Divalproex sodium | 40.1 | 35.78 | 35.78 | 34.8 | 34.8 | 34.83 | 33.92 |
| HPMC | 34.35 | — | — | — | — | — | |
| (Methocel K4 MP CR) | | | | | | | |
| HPMC (Methocel | — | 31.91 | 31.91 | 29.77 | 29.77 | 30.42 | 32.83 |
| K15 MP CR) | | | | | | | |
| HPC (Klucel EXF) | 17.16 | — | — | — | — | — | |
| HPC (Klucel HXF) | — | 6.64 | 9.97 | — | — | — | |
| Sodium Alginate | — | 16.62 | 13.29 | — | — | — | |
| (Keltone HVCR) | | | | | | | |
| Lactose (DCL 21) | — | — | — | 2.97 | 2.97 | 2.98 | 3.01 |
| MCC (Avicel PH 112) | — | — | — | 14.9 | 14.9 | 20.76 | 19.10 |
| Hydrophobic silica | 3.34 | 4.29 | 4.29 | 6.46 | 2.26 | 6.48 | 6.56 |
| (Aerosil R972) | | | | | | | |
| Colloidal Silicon | — | — | — | — | 6.46 | — | |
| dioxide (Aerosil 200) | | | | | | | |
| Magnesium Stearate | 1.94 | — | — | 1.61 | 1.61 | 1.62 | 1.6 |
| Talc | — | 1.72 | 1.72 | — | — | — | — |

Up to 3%

Procedure:
1. Divalproex sodium was sifted.
2. HPMC or Keltone HVCR and Microcrystalline cellulose were sifted.
3. Step 1 and Step 2 was mixed in blender.
4. Hydrophobic silica and magnesium Stearate were sifted.
5. Step 3 was lubricated with Step 4.
6. Step 5 was compressed to get slugs, milled and passed through sieve to get granules.
7. Microcrystalline cellulose was sifted.
8. Hydrophobic silica and Magnesium Stearate were sifted.
9. Step 7 was blended with Step 6.
10. Step 9 was lubricated with Step 8 in the blender.
11. Step 10 was compressed to get tablets and coated.

The controlled release tablets were than evaluated for hardness and friability by methods known in the art and are mentioned in Table 3

TABLE 3
Hardness and friability of formulation of the present invention

| | Trial | | | | | | |
|---|---|---|---|---|---|---|---|
| Parameters | Example 1 | Example 3 | Example 4 | Example 5 | Example 6 | Example 7 | Example 8 |
| Hardness (kp) | 13-14 | 13-14 | 15-16 | 15-16 | 15-17 | 11-12 | 15-17 |
| Friability (%) | 0.5 | 0.21 | 0.01 | 0.01 | 0.01 | 0.26 | 0.01 |

Table 4 provides the comparative dissolution profile of novel controlled release compositions of Divalproex sodium of the present invention and marketed Depakote ER.

TABLE 4
Dissolution Profile

| | Percentage of drug released | | | |
|---|---|---|---|---|
| Time (Hrs) | Depakote ER | Example 5 | Example 6 | Example 7 |
| 45 min | 8 | 5 | 6 | 6 |
| 1 | 16 | 8 | 9 | 10 |
| 3 | 28 | 20 | 19 | 20 |
| 4 | 33 | 25 | 24 | 25 |
| 6 | 42 | 35 | 33 | 36 |
| 9 | 52 | 49 | 46 | 48 |
| 12 | 63 | 61 | 58 | 59 |
| 16 | 78 | 76 | 79 | 70 |
| 18 | 93 | 87 | 92 | 75 |
| 24 | 106 | 100 | 100 | 94 |

The formulation of the present invention, as in table 4, releases not more than about 40% of total valproate after 3 hours, from about 30% to about 70% of total valproate is released after 9 hours, from about 40% to about 90% of total valproate is released after 12 hours and not less than about 75% of total valproate is released after 24 hours.

We claim:

1) A controlled release formulation comprising less than about 40% of anti-epileptic drug, about 20% to about 50% of rate controlling polymer and silica having a particle size less than about 1 micron and specific surface area not less than 70 m²/g, all weight percentages are based upon the total weight of the dosage form.

2) The formulation of claim 1 wherein the anti-epileptic drug comprise of divalproex sodium, valproic acid, sodium valproate, lamotrigine, topiramate and the like.

3) A controlled release composition comprising less than about 40% of divalproex sodium, about 20% to about 50% of rate controlling polymer and silica having a particle size less than about 1 micron free of sticking problems and specific surface area not less than 70 m²/g, all weight percentages are based upon the total weight of the dosage form.

4) The formulation of claim 3 further comprising diluent.

5) The formulation of claim 4 wherein the diluent is most preferably lactose.

6) The formulation of claim 3 comprising silica, wherein silica is hydrophilic silica or hydrophobic silica or combination of both.

7) The controlled release formulation according to claim 3, wherein the rate-controlling polymer is hydrophilic polymer and/or hydrophobic polymer.

8) The controlled release formulation according to claim 7, wherein the rate controlling polymer is hydrophilic polymer selected from hydroxypropylmethylcellulose, hydroxypropycellulose, hydroxymethycellulose, sodium alginate and the like.

9) The controlled release formulation according to claim 8, wherein the rate-controlling polymer is preferably hydroxypropylmethylcellulose.

10) The controlled release formulation according to claim 3, is prepared by wet granulation or direct compression or dry compaction and/or slugging.

11) A controlled release formulation suitable for once a day administration comprising less than about 40% of divalproex sodium, about 20% to about 50% of rate controlling polymer, silica having a particle size of less than about 1 micron and specific surface area not less than 70 m²/g, manufactured under normal atmospheric conditions, temperature of not more than about 25° C. and relative humidity of about 40% to about 55%, all weight percentages based upon the total weight of the dosage form.

12) The formulation of claim 11 further comprising diluent.

13) The formulation of claim 12 wherein the diluent is most preferably lactose.

14) The formulation of claim 11 comprising silica, wherein silica is hydrophilic silica or hydrophobic silica or combination of both.

15) The controlled release formulation according to claim 11 wherein the rate-controlling polymer is hydrophilic polymer and/or hydrophobic polymer.

16) The controlled release formulation according to claim 15, wherein the rate-controlling polymer is preferably hydroxypropylmethylcellulose.

17) The controlled release formulation according to claim 11, is prepared by wet granulation or direct compression or dry compaction and/or slugging.

18) A oral controlled release formulation of Divalproex sodium comprising:
(a) Less than about 40% of divalproex sodium,
(b) About 20% to about 50% of rate controlling polymer, preferably hydroxypropylmethylcellulose,
(c) Less than about 5% of Lactose
(d) Less than about 10% of hydrophobic silica having a particle size less than about 1 micron and specific surface area not less than 70 m²/g.
(e) Wherein said tablet exhibits the following dissolution profile, when measured in type 2 dissolution apparatus, paddle, at 100 rpm, at a temperature of 37±0.5 C, in 500 ml of 0.1N HCl for 45 min, followed by 900 ml of 0.05M phosphate buffer containing 75 mM sodium lauryl sulfate, pH 5.5 for the remainder of the testing period.
(i) Not more than about 40% of total valproate is released after 3 hours of measurement in said apparatus
(ii) From about 30% to about 70% of total valproate is released after 9 hours of measurement in said apparatus
(iii) From about 40% to about 90% of total valproate is released after 12 hours of measurement in said apparatus
(iv) Not less than about 75% of total valproate is released after 24 hours of measurement in said apparatus,
all weight percentages are based upon the total weight of the dosage form.

19) The process for preparing controlled release formulation of divalproex sodium under normal atmospheric condition, temperature of not more than about 25° C. and relative humidity of about 40% to about 55%.
